(19) United States
(12) Patent Application Publication
(10) Pub. No.: US 2010/0086453 A1
(43) Pub. Date: Apr. 8, 2010

(54) ANTISEPTIC/ANTIBACTERIAL FUMIGATOR

(76) Inventor: JASON ERIK ADAMS, ROWLETT, TX (US)

Correspondence Address: HOWISON & ARNOTT, L.L.P, P.O. BOX 741715, DALLAS, TX, 75374-1715, US

(21) Appl. No.: 12/429,293

(22) Filed: Apr. 24, 2009

Related U.S. Application Data
(60) Provisional application No. 61/195,136, filed on Oct. 3, 2008.

(51) Int. Cl. A61L 2/16

(52) U.S. Cl. 422/292

(57) ABSTRACT

A fumigation apparatus comprises a first container for holding a substance to be dispensed. The substance comprises at least two of a deodorizer, an anti-bacterial substance, an anti-allergenic substance and a disinfecting substance. A dispensing mechanism converts the substance in the first container into at least one of a mist or spray. A canister encloses the first container and the dispensing mechanism.

### CROSS-REFERENCE TO RELATED APPLICATIONS

This application claims priority from U.S. Provisional Application No. 61/195,136, filed Oct. 3, 2008 and entitled, “POOF,” which is incorporated herein by reference.

### TECHNICAL FIELD

The present invention relates to fumigators, and more particularly, to a fumigator capable of dispersing an antibacterial/deodorizing/disinfecting mist or spray.

### BACKGROUND

A fumigation device is typically used for killing pests within an enclosed area by dispensing a fumigant, which normally comprises either an insecticide for killing pests or may alternatively comprise a fragrance for deodorizing an area. While the use of fumigation devices is well-known, these devices are limited to dispensing insecticides and fragrances from the devices in order to kill pests or improve odors within a defined area. Thus, there is a need for providing a fumigation device having wider applicabilities.

### SUMMARY

The present invention as disclosed and described herein in one aspect thereof comprises a fumigation apparatus. The fumigation apparatus includes a first container for holding a substance to be dispensed. The substance includes at least two of a deodorizer, an anti-bacterial substance, anti-allergenic substance and a disinfecting substance. A dispensing mechanism converts the substance in the first container into at least one of a mist or a spray. A canister encloses the first container and the dispensing mechanism.

### BRIEF DESCRIPTION OF THE DRAWINGS

For a more complete understanding, reference is now made to the following description taken in conjunction with the accompanying Drawings in which:

FIG. 1a illustrates the fumigation device for dispensing antibacterial/deodorizing/disinfecting mist or spray;

FIG. 1b illustrates an alternative embodiment of the fumigation device of FIG. 1a;

FIG. 2 is a block diagram illustrating the basic structures included within the fumigation canister;

FIG. 3 illustrates the use of a fumigation canister within a hotel room;

FIG. 4 illustrates the various areas, which may be disinfected and deodorized using the fumigation device; and

FIG. 5 is a flow diagram describing the use of the fumigation device.

### DETAILED DESCRIPTION

Referring now to the drawings, wherein like reference numbers are used herein to designate like elements throughout, the various views and embodiments of the fumigator are illustrated and described, and other possible embodiments are described. The figures are not necessarily drawn to scale, and in some instances the drawings have been exaggerated and/or simplified in places for illustrative purposes only. One of ordinary skill in the art will appreciate the many possible applications and variations based on the following examples of possible embodiments.

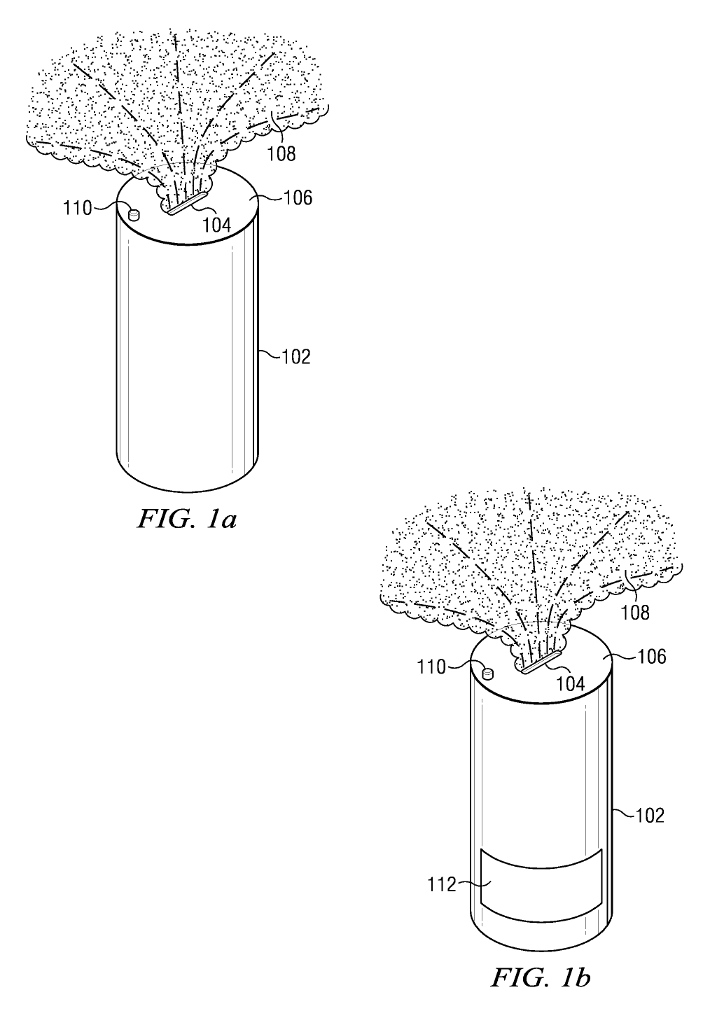

Referring now particularly FIG. 1a, there is illustrated fumigation canister 102 of the present disclosure. Fumigation canister 102 is illustrated as a cylindrical container. However, it should be realized by one skilled in the art that the container 102 of the fumigation device may comprise any geometry so long as the structure is of a sufficient size and shape to contain the components for dispensing the mist or spray from the container 102 as described hereinbelow. Defined on the container 102 is a slot 104 within the top surface 106 of the container 104. The slot 104 is used for expelling the fumigant from the interior of the cylindrical container 102. While the slot 104 is illustrated as being within the top surface 106 of the cylindrical container 102, it will be appreciated that the slot 104 could be located on any portion of the cylindrical container 102. However, in most cases, the slot 104 will be located toward the top of the container 102 as the mist or spray created within the container 102 will rise toward the top of the container 102 and a slot located on the upper portion of the container 102 will facilitate the expulsion of the mist or spray from the container 102. Additionally, rather than a single slot 104 as illustrated in FIG. 1, multiple slots may be utilized or alternatively holes, grids, grates or other types of openings may be utilized for providing an opening for the mist or spray of the fumigator to exit the device. In other embodiments, a spray nozzle or jet dispenser may also be used.

The device dispenses the material from within the container 102 as a mist or spray as shown generally at 108. The mist and/or spray 108 may comprise the combination of antibacterial solution, a deodorizing solution, anti-allergen solution and/or a disinfecting solution. Thus, rather than merely providing a pleasant fragrance within the mist/spray, the mist/spray will provide an antibacterial solution that is useful in killing germs and a disinfecting material for cleaning surfaces and areas with which the spray may come in contact. The combination of the disinfectants/antibacterial/anti-allergen/deodorizer within the same device enables a fumigation device that quickly disinfects and deodorizers a desired area. The materials used within the mist/spray 108 are quick-drying such that once the mist or spray comes in contact with a surface; it would dry within a relatively fast period of time. This would prevent the creation of a wet buildup that lingered on the surface of items within a room being exposed to the fumigator and provide an undesired wet surface. The antibacterial portion of the spray 108 will disinfect the area by killing germs within the entire area while at the same time providing the deodorizing benefits of a fragrance.

The components used within the mist/spray would also have to be non-toxic, non-filming, non-flammable, fast-drying, pet and child friendly, and provide multiple fragrances depending upon a user's desire. The use of non-toxic materials is necessary to prevent undesired poisoning or side effects caused from the use of the fumigation canister within areas frequented by humans. The non-filming characteristics of the mist would be desirable as the fumigator could be used multiple times within for example, a hotel room, hospital or airplane, and by having non-filming characteristics, there would be no need for an individual to come back after multiple uses of the device to clean the film created by the fumigation device from the surfaces within the area being treated. Non-flammable characteristics are desired to prevent flash fires or explosions being caused by use of the fumigation device within an enclosed area. The fast-drying characteristics of the mist would enable the fumigation device to be quickly used within an area that was to be shortly occupied by individuals. Pet and child friendly characteristics are necessary for use within areas that may be frequented by children and/or pets. Multiple fragrances provide a consumer options that allow individuals to select a particular scent that they find pleasing when taking benefit of the antibacterial/and disinfecting characteristics of the device.

In a preferred embodiment, the canister 102 is a portable device that may be easily moved to any location within a desired area of treatment. Furthermore, the product is envisioned as a one time disposable device, wherein once the device has been actuated and used to disinfect a particular area it may be removed and disposed of. The canister would be of varying sizes depending on a desired area of treatment. Larger volume canisters could be used to treat larger areas. Alternatively, the device could include a refillable reservoir as illustrated in FIG. 1B. In this embodiment, the opening or slot 112 provides access to a refillable reservoir may be opened in order to add additional disinfectants/antibacterial/anti-allergen/deodorizer material within the canister 102. Once this has been done, the opening or slot 112 is closed enabling the device to again be actuated.

Actuation of the device may be accomplished via an actuation button or switch 110. The button or switch activates the mechanical components contained within the fumigation container 102 in order to dispense the antibacterial/disinfectants/anti-allergen/deodorizing material contained within the device.

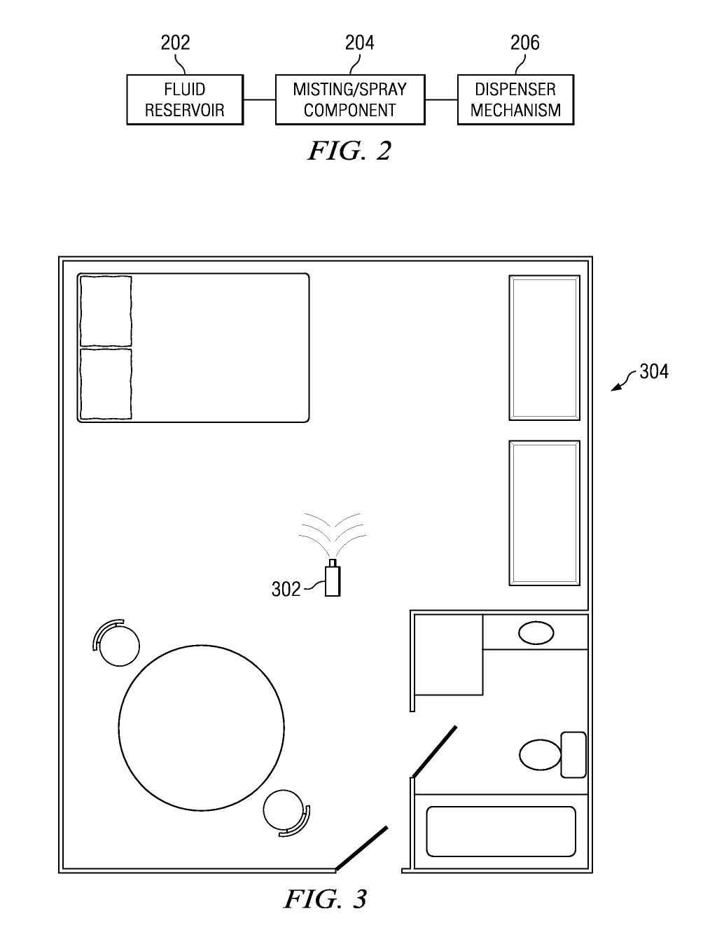

Referring now to FIG. 2, there is illustrated a functional block diagram of the internal components contained within the fumigation canister 102 of FIG. 1. A fluid reservoir 202 contains the antibacterial/disinfectants/anti-allergen/deodorizer material. The fluid reservoir 202 may contain each of these components that are mixed together within a single chamber, or alternatively, separate chambers for each of the antibacterial/disinfectants/anti-allergen/deodorizer fluids may be utilized within the fluid reservoir 202. Each material could then be provided in a desired percentage based upon the use. While the present description envisions the use of a fluid material for dispensing by the device, it will be appreciated by one skilled in the art that other components of a solid or gel nature may be utilized that provide the antibacterial/disinfecting/deodorizing benefits from the fumigation canister in a similar fashion. Thus, rather than a fluid reservoir 202, a container for containing solid or gel or any other state material may be utilized.

Connected with the fluid reservoir 202 is a misting/spray component 204. The misting/spray component 204 takes the fluid, solid, etc. material from the reservoir 202 and converts it into a mist and/or spray state that is useful for dispensing the material from the fumigation device. The particular components used for creating the misting/spray state of the material may comprise any number of embodiments. However, in keeping with the desires of the fumigation device, these components would need to be self-contained, capable of operating responsive to a mechanical power source, chemically actuated power source or a battery such that the device is capable of being self-contained and portable within the desired area of treatment.

Finally, the dispenser mechanism 206 comprises components for expelling the mist/spray from the fumigation canister with sufficient force such that the mist/spray is able to provide a wide area of treatment within the location that the device has been placed. This may comprise some type of spray nozzle or slot for enabling expulsion of the spray/mist as discussed previously with respect to FIG. 1.

Referring now to FIG. 3, there is illustrated the use of the fumigation device 302 within a hotel room 304. By placing one or more fumigation devices 302 within the hotel room 304, a large area of effect may be disinfected/deodorized and treated with an antibacterial material. Thus, with one or more containers, the beds, furniture, tables, bathroom, closets, rugs, etc. may be quickly treated and disinfected after use of one customer of the hotel prior to use by a next customer.

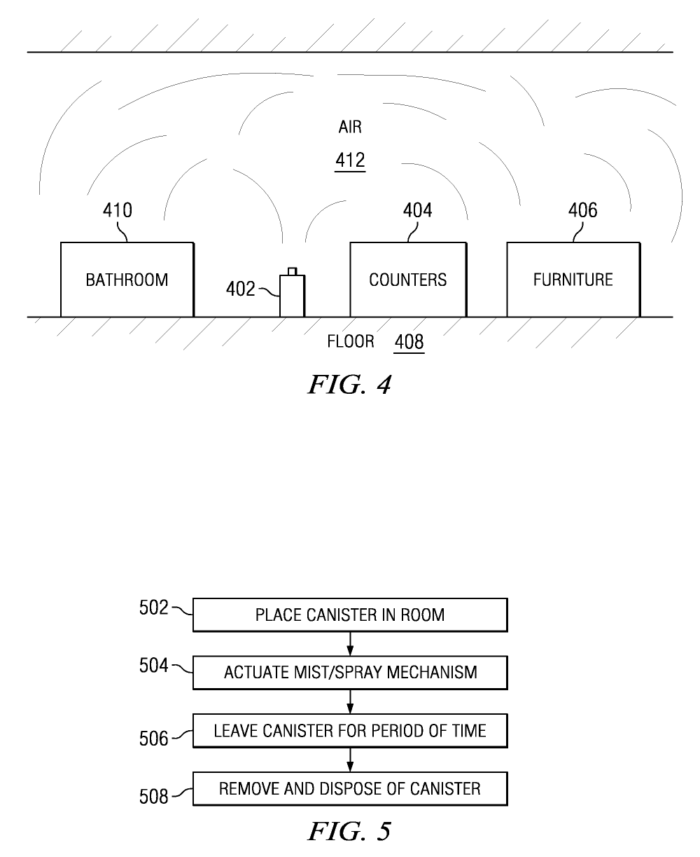

It can of course be appreciated that use of the fumigation canister 302 may be used within a variety of areas other than hotels, hotel and motel rooms. The device would find useful applications within apartments and homes. This is illustrated in FIG. 4, wherein a single canister may be used to treat a number of areas within a home including kitchen and bathroom counters 404, furniture within the home 406, floors of the home 408, bathroom areas 410 and the air 412 of the home. Thus, a single canister 402 may have a wide area of affect to treat a number of areas that normally would have to be individually treated and cleaned using spot spraying and hand wiping techniques. This may be useful in homes for daily cleaning, special cleanings after illnesses within the home, people who are germophobic, etc.

Other areas which may find useful application of the fumigation canister include schools, which are known to provide breeding grounds for germs due to the large number of children present. The ability to daily or weekly disinfect the school using the fumigation device would eliminate some of these germs that might be transferred from person to person. Another area of application might be within hospital rooms between patient stays. Additionally, waiting areas where ill patients may wait before treatment, might benefit from treatment using the fumigation device. Airplanes comprise another area of treatment. After a flight, the fumigation device could be utilized within the airplane to eliminate germs that would be present due to the confined contact of a large number of people. Other areas such as office buildings would also benefit from treatments on a daily/weekly basis using the described fumigation device. Practically any enclosed, high-traffic area where germs and/or smells may linger could potentially benefit from use of the described fumigation device providing an antibacterial/disinfectants/anti-allergen/deodorizing spray.

Referring now to FIG. 5, there is illustrated a flow diagram describing the general method of use of the disinfecting fumigator device of the present disclosure. Initially, the canister containing the fumigation device is placed at step 502 within a room or area of treatment that is to benefit from the operation of the device. Next, the mist/spray mechanism is actuated at step 504 to start the mist and/or spray from being dispensed from the fumigation canister and begin treating the desired area. The canister is left within the area at step 506 for a necessary period of time necessary for the contents of the canister to be completely dispensed within the room. The period of time necessary for the canister to completely dispense its contents will depend upon the size of the canister and the particular dispensing mechanism which is being utilized. Finally, once the contents of the canister have been completely dispensed within the room providing its antibacterial/disinfectants/anti-allergen/deodorizing effects, the canister may be removed and disposed of at step 508.

Thus, the described fumigation device provides a safe and effective antibacterial and/or disinfectant and/or antiallergen/hypoallergenic and/or scented or unscented deodorizing fumigator. The disinfecting and/or antiallergen/hypoallergenic and/or deodorizing and/or antibacterial is contained within the fumigation canister. The dispensing canister can be placed within a needed area of a room. Once the container is activated and spray/disperses the product throughout the room or other area, the need to stand and spray and clean an area by hand is eliminated.

It will be appreciated by those skilled in the art having the benefit of this disclosure that this antiseptic/antibacterial fumigator provides an improved apparatus and method for cleaning and treating high-traffic enclosed areas. It should be understood that the drawings and detailed description herein are to be regarded in an illustrative rather than a restrictive manner, and are not intended to be limiting to the particular forms and examples disclosed. On the contrary, included are any further modifications, changes, rearrangements, substitutions, alternatives, design choices, and embodiments apparent to those of ordinary skill in the art, without departing from the spirit and scope hereof, as defined by the following claims. Thus, it is intended that the following claims be interpreted to embrace all such further modifications, changes, rearrangements, substitutions, alternatives, design choices, and embodiments.

What is claimed is:

1. A fumigation apparatus, comprising:
a first container for holding a substance to be dispensed, the substance comprising at least two of a deodorizer, an anti-bacterial substance, an anti-allergen substance and a disinfecting substance;
a dispensing mechanism for converting the substance in the first container into at least one of a mist or spray; and
a canister for enclosing the first container and the dispensing mechanism.

2. The fumigation apparatus of claim 1, wherein the dispensing mechanism further comprises:
a mist generator for converting the substance into a mist; and
an output device for releasing the mist from the canister.

3. The fumigation apparatus of claim 1, wherein the dispensing mechanism further comprises:
a spray generator for generating a spray from the substance; and
a spray nozzle for dispensing the spray responsive to the spray from the spray generator.

4. The fumigation apparatus of claim 1, wherein the substance comprises a quick drying solution.

5. The fumigation apparatus of claim 1, wherein the substance comprises a non-filming solution.

6. The fumigation apparatus of claim 1, wherein the apparatus comprise a one time use device that is disposable after use.

7. The fumigation device of claim 1, wherein the first container is refillable to replenish the substance.

8. The fumigation device of claim 1, wherein the first container includes separate chambers for containing each of the at least two of the deodorizer, the anti-bacterial substance, the anti-allergen substance and the disinfecting substance.

9. A fumigation apparatus, comprising:
a first container for holding a substance to be dispensed, the substance comprising a deodorizer and an anti-bacterial substance;
a dispensing mechanism for converting the substance in the first container into at least one of a mist or spray; and
a canister for enclosing the first container and the dispensing mechanism.

10. The fumigation apparatus of claim 9, wherein the substance further includes a disinfecting substance.

11. The fumigation apparatus of claim 9, wherein the dispensing mechanism further comprises:
a mist generator for converting the substance into a mist; and
an output device for releasing the mist from the canister.

12. The fumigation apparatus of claim 9, wherein the dispensing mechanism further comprises:
a spray generator for generating a spray from the substance; and
a spray nozzle for dispensing the spray responsive to the spray from the spray generator.

13. The fumigation apparatus of claim 9, wherein the substance comprises a quick drying solution.

14. The fumigation apparatus of claim 9, wherein the substance comprises a non-filming solution.

15. A fumigation apparatus, comprising:
a first container for holding a substance to be dispensed, the substance comprising a deodorizer and a disinfecting substance;
a dispensing mechanism for converting the substance in the first container into at least one of a mist or spray; and
a canister for enclosing the first container and the dispensing mechanism.

16. The fumigation apparatus of claim 15, wherein the substance further includes an anti-bacterial substance.

17. The fumigation apparatus of claim 15, wherein the dispensing mechanism further comprises:
a mist generator for converting the substance into a mist; and
an output device for releasing the mist from the canister.

18. The fumigation apparatus of claim 15, wherein the dispensing mechanism further comprises:
a spray generator for generating a spray from the substance; and
a spray nozzle for dispensing the spray responsive to the spray from the spray generator.

19. The fumigation apparatus of claim 15, wherein the substance comprises a quick drying solution.

20. The fumigation apparatus of claim 15, wherein the substance comprises a non-filming solution.
